(19) United States
(12) Patent Application Publication
(10) Pub. No.: US 2024/0052423 A1
(43) Pub. Date: Feb. 15, 2024

(54) METHODS OF IDENTIFYING A TUMOR THAT IS SENSITIVE TO TREATMENT WITH TALAZOPARIB AND METHODS OF TREATMENT THEREOF

(71) Applicant: PFIZER INC., NEW YORK, NY (US)

(72) Inventors: Joshua James GRUBER, Dallas, CA (US); Melinda TELLI, San Francisco, CA (US)

(73) Assignee: PFIZER INC., NEW YORK, NY (US)

(21) Appl. No.: 18/265,656

(22) PCT Filed: Dec. 6, 2021
(86) PCT No.: PCT/IB2021/061372

§ 371 Date: Jun. 6, 2023

Related U.S. Application Data
(60) Provisional application No. 63/122,453, filed on Dec. 7, 2020.

(51) Int. Cl. C12Q 1/6886

(52) U.S. Cl. CPC C12Q 1/6886; C12Q 2600/156

(57) ABSTRACT

The present invention relates to a method of identifying a metastatic tumor determined to have a mutation in homologous recombination pathway genes, that is sensitive to treatment with talazoparib, or a pharmaceutically acceptable salt thereof, and methods of treatment thereof, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2. The present invention also relates to a method of selecting a subject determined to have a metastatic tumor with a mutation in homologous recombination pathway genes, for treatment with talazoparib, or a pharmaceutically acceptable salt thereof.

### FIELD OF THE INVENTION

The present invention relates to methods of identifying a tumor that is sensitive to treatment with talazoparib and methods of treatment thereof. The present invention also relates to methods of selecting a subject for treatment with talazoparib.

### BACKGROUND

Poly (ADP-ribose) polymerase (PARP) engages in the naturally occurring process of DNA repair in a cell. PARP inhibition has been shown to be an effective therapeutic strategy against tumors associated with germline mutation in double-strand DNA repair genes by inducing synthetic lethality (Sonnenblick, A., et al., Nat. Rev. Clin. Oncol, 2015, 12(1), 27-4).

Talazoparib is a potent, orally available small molecule PARP inhibitor, which is cytotoxic to human cancer cell lines harboring gene mutations that compromise deoxyribonucleic acid (DNA) repair, an effect referred to as synthetic lethality, and by trapping PARP protein on DNA thereby preventing DNA repair, replication, and transcription.

The compound, talazoparib, which is “(8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one” and “(8S,9R)-5-fluoro-8-(4-fluorophenyl)-9-(1-methyl-1H-1,2,4-triazol-5-yl)-2,7,8,9-tetrahydro-3H-pyrido[4,3,2-de]phthalazin-3-one” (also referred to as “PF-06944076”, “MDV3800”, and “BMN673”) is a PARP inhibitor, having the structure,

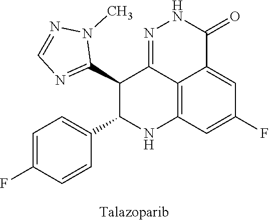 [SMILES: Cn1ncnc1[C@H]1c2n[nH]c(=O)c3cc(F)cc(c23)N[C@@H]1c1ccc(F)cc1]

Talazoparib, and pharmaceutically acceptable salts thereof, including the tosylate salt, are disclosed in International Publication Nos. WO 2010/017055 and WO 2012/054698. Additional methods of preparing talazoparib, and pharmaceutically acceptable salts thereof, including the tosylate salt, are described in International Publication Nos. WO 2011/097602, WO 2015/069851, and WO 2016/019125. Additional methods of treating cancer using talazoparib, and pharmaceutically acceptable salts thereof, including the tosylate salt, are disclosed in International Publication Nos. WO 2011/097334 and WO 2017/075091.

TALZENNA® (talazoparib) (0.25 mg and 1 mg capsules) has been approved in several countries, including the United States, and in the European Union, and is approved or under review with anticipated approvals in other countries for the treatment of adult patients with deleterious or suspected deleterious gBRCAm HER2-negative locally advanced or metastatic breast cancer. Talazoparib is under development for a variety of human cancers both as a single agent and in combination with other agents. Additional capsule strengths, 0.5 mg and 0.75 mg, have been approved in the United States.

Talazoparib is active in gBRCAm HER2-negative locally advanced or metastatic breast cancer; however, there is a need to identify and select cancer patients who may respond to talazoparib treatment beyond patients having BRCA1 and/or BRCA2 mutated cancers.

### SUMMARY

Each of the embodiments of the present invention described below may be combined with one or more other embodiments of the present invention described herein which is not inconsistent with the embodiment(s) with which it is combined. In addition, each of the embodiments below describing the invention envisions within its scope the pharmaceutically acceptable salts of the compounds of the invention.

The present invention relates to a method of identifying a metastatic tumor determined to have a mutation in homologous recombination pathway genes, that is sensitive to treatment with talazoparib, or a pharmaceutically acceptable salt thereof, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

One embodiment of the present invention relates to the method of identifying a metastatic tumor determined to have a mutation in homologous recombination pathway genes, wherein the mutation is a somatic or a germline mutation.

One embodiment of the present invention relates to the method of identifying a metastatic tumor determined to have a mutation in homologous recombination pathway genes, wherein the mutation is PALB2, CHEK2, ATM, BR1P1, RAD50, ATR, PTEN, or FANCA.

One embodiment of the present invention relates to the method of identifying a metastatic tumor determined to have a mutation in homologous recombination pathway genes, wherein the mutation is gCHEK2, gPALB2, or sPTEN.

One embodiment of the present invention relates to the method of identifying a metastatic tumor determined to have a mutation in homologous recombination pathway genes, wherein the mutation is gPALB2.

One embodiment of the present invention relates to the method of identifying a metastatic tumor, wherein the metastatic tumor is a breast tumor, and further wherein the breast tumor is a HER2-negative breast tumor.

One embodiment of the present invention relates to the method of identifying a metastatic tumor determined to have a mutation in homologous recombination pathway genes, wherein the metastatic tumor is determined to have a mutation in homologous recombination pathway genes by next generation sequencing.

One embodiment of the present invention relates to the method of identifying a metastatic tumor determined to have a mutation in homologous recombination pathway genes, wherein the step of determining a homologous recombination deficiency score from a biopsy of the metastatic tumor is performed by next generation sequencing.

One embodiment of the present invention relates to the method of identifying a metastatic tumor determined to have a mutation in homologous recombination pathway genes, wherein the homologous recombination deficiency score is at least 42%.

One embodiment of the present invention relates to a method of treating metastatic cancer comprising administering talazoparib, or a pharmaceutically acceptable salt thereof, according to any one of the previous embodiments.

The present invention also relates to a method of selecting a subject determined to have a metastatic tumor with a mutation in homologous recombination pathway genes, for treatment with talazoparib, or a pharmaceutically acceptable salt thereof, comprising a) determining a homologous recombination deficiency score from a biopsy of the metastatic tumor; and b) selecting the subject for treatment with talazoparib, or a pharmaceutically acceptable salt thereof, if the homologous recombination deficiency score is at least 33%, wherein the mutation is not germline BRCA1 or germline BRCA2.

One embodiment of the present invention further comprises administering talazoparib, or a pharmaceutically acceptable salt thereof, to the selected patient.

One embodiment of the present invention relates to the method of selecting a subject determined to have a metastatic tumor with a mutation in homologous recombination pathway genes, wherein the mutation is a somatic or a germline mutation.

One embodiment of the present invention relates to the method of selecting a subject determined to have a metastatic tumor with a mutation in homologous recombination pathway genes, wherein the mutation is PALB2, CHEK2, ATM, BR1P1, RAD50, ATR, PTEN, or FANCA.

One embodiment of the present invention relates to the method of selecting a subject determined to have a metastatic tumor with a mutation in homologous recombination pathway genes, wherein the mutation is gCHEK2, gPALB2, or sPTEN.

One embodiment of the present invention relates to the method of selecting a subject determined to have a metastatic tumor with a mutation in homologous recombination pathway genes, wherein the mutation is gPALB2.

One embodiment of the present invention relates to the method of selecting a subject determined to have a metastatic tumor with a mutation in homologous recombination pathway genes, wherein the metastatic tumor is a breast tumor, and further wherein the breast tumor is a HER2-negative breast tumor.

One embodiment of the present invention relates to the method of selecting a subject determined to have a metastatic tumor with a mutation in homologous recombination pathway genes, wherein the metastatic tumor is determined to have a mutation in homologous recombination pathway genes by next generation sequencing.

One embodiment of the present invention relates to the method of of selecting a subject determined to have a metastatic tumor with a mutation in homologous recombination pathway genes, wherein the step of determining a homologous recombination deficiency score from a biopsy of the metastatic tumor is performed by next generation sequencing.

One embodiment of the present invention relates to the method of identifying a metastatic tumor with a mutation in homologous recombination pathway genes, wherein the homologous recombination deficiency score is at least 42%.

The present invention relates to a method of treating a metastatic tumor in a subject determined to have a mutation in homologous recombination pathway genes, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

One embodiment of the present invention relates to the method of treating metastatic cancer in a subject determined to have a mutation in homologous recombination pathway genes, wherein the mutation is a somatic or a germline mutation.

One embodiment of the present invention relates to the method of treating metastatic cancer in a subject determined to have a mutation in homologous recombination pathway genes, wherein the mutation is PALB2, CHEK2, ATM, BRIP1, RAD50, ATR, PTEN, or FANCA.

One embodiment of the present invention relates to the method of treating metastatic cancer in a subject determined to have a mutation in homologous recombination pathway genes, wherein the mutation is gCHEK2, gPALB2, or sPTEN.

One embodiment of the present invention relates to the method of treating metastatic cancer in a subject determined to have a mutation in homologous recombination pathway genes, wherein the mutation is gPALB2.

One embodiment of the present invention relates to the method of treating metastatic cancer in a subject determined to have a mutation in homologous recombination pathway genes, wherein the metastatic tumor is a breast tumor.

One embodiment of the present invention relates to the method of treating metastatic cancer in a subject determined to have a mutation in homologous recombination pathway genes, wherein the breast tumor is a HER2-negative breast tumor.

One embodiment of the present invention relates to the method of treating metastatic cancer in a subject determined to have a mutation in homologous recombination pathway genes, wherein the metastatic tumor is determined to have a mutation in homologous recombination pathway genes by next generation sequencing.

One embodiment of the present invention relates to the method of treating metastatic cancer in a subject determined to have a mutation in homologous recombination pathway genes, wherein the step of determining a homologous recombination deficiency score from a biopsy of the metastatic tumor is performed by next generation sequencing.

One embodiment of the present invention relates to the method of treating metastatic cancer in a subject determined to have a mutation in homologous recombination pathway genes, wherein the homologous recombination deficiency score is at least 42%.

### BRIEF DESCRIPTION OF THE DRAWINGS

FIG. 1 shows the flow diagram of a phase II clinical trial of talazoparib in BRCA1 and BRCA2 wild-type patients with advanced HER2-negative breast cancer or other solid tumors with a mutation in homologous recombination.

FIG. 2 shows the best treatment response for all patients in a waterfall plot of best change in Sum of Longest Diameters (SLD) of target lesions by RECIST v.1.1 for all treated patients (n=20) by tumor type. Germline (g) or somatic (s) mutations in genes used for enrollment are indicated. Dashed line represents 30% decrease in tumor size.

FIG. 3 shows the plot of homologous recombination deficiency (HRD) scores in primary and metastatic samples for all evaluable patients. Horizontal dotted lines indicate HRD threshold of 33 (captures 99% of known BRCA1/2-deficient ovarian cancers) or 42 (captures 95% of known BRCA1/2-deficient ovarian cancers).

FIG. 4 shows the plot of HRD scores for paired primary and metastatic samples. Horizontal dotted lines indicate HRD threshold of 33 (captures 99% of known BRCA1/2-deficient ovarian cancers) or 42 (captures 95% of known BRCA1/2-deficient ovarian cancers.

FIG. 5 shows the plot of HRD score against best change in SLD by RECIST. Pearson's correlation (r=0.64; p=0.008) is indicated by solid line. Vertical dotted lines indicate HRD threshold of ≥33 (captures 99% of known BRCA112-deficient ovarian cancers) or ≥42 (captures 95% of known BRCA1/2-deficient ovarian cancers). Tumor types are labeled by gene mutation used for enrollment. In cases where patients had more than one HRD score (eg. due to assay of primary and metastatic tumors) the higher score was used.

### DETAILED DESCRIPTION

The present invention may be understood more readily by reference to the following detailed description of the preferred embodiments of the invention. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” plasticizer includes one or more plasticizers.

As used herein, the term “about” when used to modify a numerically defined parameter (e.g., an amount of talazoparib) means that the parameter may vary by as much as 10% below or above the stated numerical value for that parameter. For example, a dose of about 1 mg may vary between 0.9 mg and 1.1 mg.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

The terms “cancer”, “cancerous”, and “malignant” refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. Examples of cancer include, but are not limited to, carcinoma, lymphoma, leukaemia, blastoma, and sarcoma. More particular examples of such cancers include squamous cell carcinoma, myeloma, lung cancer, small-cell lung cancer, small cell prostate cancer, non-small cell lung cancer, glioma, hodgkin's lymphoma, non-hogkin's lymphoma, follicular lymphoma (FL), diffuse large B-cell lymphoma (DLCBCL), acute myeloid leukaemia (AML), multiple myeloma, gastrointestinal (tract) cancer, renal cancer, ovarian cancer, uterine cancer, endometrial cancer, liver cancer, kidney cancer, renal cell carcinoma, prostate cancer, castration-sensitive prostate cancer, castration-resistant prostate cancer (CRPC), thyroid cancer, melanoma, chondrosarcoma, neuroblastoma, pancreatic cancer, glioblasoma, multiformer, cervical cancer, rectal cancer, brain cancer, stomach cancer, bladder cancer, hepatoma, hepatocellular carcinoma, breast cancer, colon cancer, head and neck cancer, and salivary gland cancer.

The term “patient” or “subject” refers to any single subject for which therapy is desired or that is participating in a clinical trial, epidemiological study or used as a control, including humans and mammalian veterinary patients such as cattle, horses, dogs and cats. In certain preferred embodiments, the subject is a human.

The term “treat” or “treating” a cancer as used herein means to administer a therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cells; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (200)). In some embodiments, the treatment achieved by a method of the invention is any of the partial response (PR), complete response (CR), overall response (OR), objective response rate (ORR), progression free survival (PFS), radiographic PFS, disease free survival (DFS) and overall survival (OS). PFS, also referred to as “Time to Tumor Progression” indicates the length of time during and after treatment that the cancer does not grow, and includes the amount of time patients have experience a CR or PR, as well as the amount of time patients have experience stable disease (SD). DFS refers to the length of time during and after treatment that the patient remains free of disease. OS refers to a prolongation in life expectancy as compared to naïve or untreated subjects or patients. In some embodiments, response to a method of the invention is any of PR, CR, PFS, DFS, ORR, OR or OS. Response to a method of the invention, including duration of soft tissue response, is assessed using Response Evaluation Criteria in Solid Tumors version 1.1 (RECIST 1.1) response criteria. In some embodiments, the treatment achieved by a method of the invention is measured by the time to PSA progression, the time to initiation of cytotoxic chemotherapy and the proportion of patients with PSA response greater than or equal to 50%. The treatment regimen for a method of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as, but not limited to, the Cox log-rank test, the Cochran-Mantel-Haenszel log-rank test, the Student's t-test, the chi2-test, the U-test according to Mann and Whitney, the Kruskal-Wallis test (H-test), Jonckheere-Terpstrat-test and the Wilcon on-test. The term “treatment” also encompasses in vitro and ex vivo treatment, e.g., of a cell, by a reagent, diagnostic, binding compound, or by another cell.

As used herein, a “dosage”, an “amount”, an “effective dosage” or “effective amount” of drug, compound or pharmaceutical formulation is an amount sufficient to effect any one or more beneficial or desired, including biochemical, histological and/or behavioral symptoms, of the disease, its complications and intermediate pathological phenotypes presenting during development of the disease. For therapeutic use, a “therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or delaying the progression of the disease of patients. An effective dosage can be administered in one or more administrations. For the purposes of this invention, an effective dosage of drug, compound, or pharmaceutical formulation is an amount sufficient to accomplish prophylactic or therapeutic treatment either directly or indirectly. As is understood in the clinical context, an effective dosage of drug, compound or pharmaceutical formulation may or may not be achieved in conjunction with another drug, compound or pharmaceutical formulation.

In an embodiment, an amount of talazoparib, or a pharmaceutically acceptable salt thereof, and preferably a tosylate thereof, is administered at a daily dosage of from about 0.1 mg to about 2 mg once a day, preferably from about 0.25 mg to about 1.5 mg once a day, and more preferably from about 0.5 mg to about 1.0 mg once a day. In an embodiment, talazoparib or a pharmaceutically acceptable salt thereof, and preferably a tosylate thereof, is administered at a daily dosage of about 0.1 mg, about 0.25 mg, about 0.35 mg, about 0.5 mg, about 0.75 mg or about 1.0 mg once daily. In an embodiment, talazoparib or a pharmaceutically acceptable salt thereof, and preferably a tosylate thereof, is administered at a daily dosage of about 0.1 mg, about 0.25 mg, about 0.35 mg, or about 0.5 mg once daily. In an embodiment, talazoparib or a pharmaceutically acceptable salt thereof, and preferably a tosylate thereof, is administered at a daily dosage of about 0.25 mg, about 0.35 mg, or about 0.5 mg once daily. In an embodiment, talazoparib or a pharmaceutically acceptable salt thereof and preferably a tosylate thereof, is administered at a daily dosage of about 0.5 mg, about 0.75 mg or about 1.0 mg once daily. In an embodiment, talazoparib or a pharmaceutically acceptable salt thereof and preferably a tosylate thereof, is administered at a daily dosage of about 0.1 mg once daily. In an embodiment, talazoparib or a pharmaceutically acceptable salt thereof, and preferably a tosylate thereof, is administered at a daily dosage of about 0.25 mg once daily. In an embodiment, talazoparib or a pharmaceutically acceptable salt thereof, and preferably a tosylate thereof, is administered at a daily dosage of about 0.35 mg once daily. In an embodiment, talazoparib or a pharmaceutically acceptable salt thereof, and preferably a tosylate thereof, is administered at a daily dosage of about 0.5 mg once daily. In an embodiment, talazoparib or a pharmaceutically acceptable salt thereof, and preferably a tosylate thereof, is administered at a daily dosage of about 0.75 mg once daily. In an embodiment, talazoparib or a pharmaceutically acceptable salt thereof, and preferably a tosylate thereof, is administered at a daily dosage of about 1.0 mg once daily. Dosage amounts provided herein refer to the dose of the free base form of talazoparib or are calculated as the free base equivalent of an administered talazoparib salt form. For example, a dosage or amount of talazoparib, such as 0.5, 0.75 mg or 1.0 mg refers to the free base equivalent. This dosage regimen may be adjusted to provide the optimal therapeutic response. For example, the dose may be proportionally reduced or increased as indicated by the exigencies of the therapeutic situation.

The term “pharmaceutically acceptable salt”, as used herein, unless otherwise indicated, refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

“Tumor burden” also referred to as a “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g., using callipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using callipers, or while in the body using imaging techniques, e.g., bone scan, ultrasound, CR or MRI scans.

The methods of the present invention are useful for treating cancer. Additionally, the methods of the present invention are useful for identifying a metastatic tumor determined to have a mutation in homologous recombination pathway genes, that is sensitive to cancer treatment, such as treatment with talazoparib. In some embodiments, the methods provided results in one or more of the following effects: (1) inhibiting cancer cell proliferation; (2) inhibiting cancer cell invasiveness; (3) inducing apoptosis of cancer cells; (4) inhibiting cancer cell metastasis; (5) inhibiting angiogenesis; or (6) overcoming one or more resistance mechanisms relating to a cancer treatment.

According to the methods of the present invention, a metastatic tumor may be determined to have a mutation in homologous recombination pathway genes using next generation sequencing.

As known in the art, a “homologous recombination deficiency score” or “(HRD) score” integrates three DNA-based measures of genomic instability and is defined as the sum of loss-of-heterozygosity, telomeric allelic imbalance, and large-scale state transitions.

According to the methods of the present invention, a homologous recombination deficiency score from a biopsy of a metastatic tumor may be determined using next generation sequencing (NGS). For example, a homologous recombination deficiency (HRD) assay, such as myChoice® CDx (Myriad Genetics, Inc.) may be utilized. The myChoice® CDx is the first and only FDA-approved tumor test that determines homologous recombination deficiency status by detecting BRCA1 and BRCA2 (sequencing and large rearrangement) variants and assessing genomic instability using three critical biomarkers: loss of heterozygosity, telomeric allelic imbalance and large-scale state transitions. Myriad myChoice® CDx is a next generation sequencing-based in vitro diagnostic test that assesses the qualitative detection and classification of single nucleotide variants, insertions and deletions, and large rearrangement variants in protein coding regions and intron/exon boundaries of the BRCA1 and BRCA2 genes and the determination of Genomic Instability Score (GIS) which is an algorithmic measurement of Loss of Heterozygosity (LOH), Telomeric Allelic Imbalance (TAI), and Large-scale State Transitions (LST) using DNA isolated from formalin-fixed paraffin embedded (FFPE) tumor tissue specimens.

In an embodiment, this invention relates to method of treating metastatic cancer in a subject determined to have a mutation in homologous recombination pathway genes, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

In another aspect, this invention relates to a use of talazoparib, or a pharmaceutically acceptable salt thereof, in the treatment of metastatic cancer in a subject determined to have a mutation in homologous recombination pathway genes, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

In another aspect, this invention relates to a use of talazoparib, or a pharmaceutically acceptable salt thereof, as a medicament for the treatment of metastatic cancer in a subject determined to have a mutation in homologous recombination pathway genes, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

In one embodiment of the invention, the subject is a mammal.

In one embodiment of the invention, the subject is a human.

In some embodiments the methods of the present invention may be useful for the treatment of cancers including but not limited to cancers of the:
circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue;
respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);
genitourinary tract, for example, kidney (adenocarcinoma, Wilm's tumor [nephroblastoma], lymphoma, leukemia), bladder and/or urethra (squamous cell carcinoma, transitional cell or urothelial carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
liver (for example, hepatoma, hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma);
bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs;
hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];
oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx;
skin, for example, malignant melanoma, cutaneous melanoma, basal cell carcinoma, squamous cell carcinoma, Karposi's sarcoma, moles dysplastic nevi, lipoma, angioma, dermatofibroma, and keloids;
adrenal glands: neuroblastoma; and
other tissues including connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

In one embodiment, examples of “cancer” when used herein in connection with the present invention include cancer selected from lung cancer (NSCLC and SCLC), breast cancer (including triple negative breast cancer, hormone positive breast cancer, HER2 negative breast cancer, HER2 positive breast cancer and triple positive breast cancer), ovarian cancer, colon cancer, rectal cancer, cancer of the anal region, prostate cancer (including castration-sensitive or hormone sensitive prostate cancer and hormone-refractory prostate cancer, also known as castration-resistant prostate cancer), hepatocellular carcinoma, diffuse large B-cell lymphoma, follicular lymphoma, melanoma and salivary gland tumor or a combination of one or more of the foregoing cancers.

In one embodiment, examples of “cancer” when used herein in connection with the present invention include cancer selected from lung cancer (NSCLC and SCLC), breast cancer (including triple negative breast cancer, hormone positive breast cancer, and HER2 negative breast cancer), ovarian cancer, prostate cancer (including castration-sensitive or hormone sensitive prostate cancer and hormone-refractory prostate cancer, also known as castration-resistant prostate cancer), or a combination of one or more of the foregoing cancers.

In one embodiment, examples of “cancer” when used herein in connection with the present invention include cancer selected from prostate cancer, androgen receptor positive breast cancer, hepatocellular carcinoma, and salivary gland tumor, or a combination of one or more of the foregoing cancers.

In one embodiment, examples of “cancer” when used herein in connection with the present invention include cancer selected from androgen receptor positive breast cancer, hepatocellular carcinoma, and salivary gland tumor, or a combination of one or more of the foregoing cancers.

In one embodiment, examples of “cancer” when used herein in connection with the present invention include cancer selected from triple negative breast cancer, hormone positive breast cancer, HER2 negative breast cancer, triple positive breast cancer, castration-sensitive prostate cancer, castration-resistant prostate cancer, hepatocellular carcinoma, and salivary gland tumor or a combination of one or more of the foregoing cancers.

In one embodiment, examples of “cancer” when used herein in connection with the present invention include cancer selected from triple negative breast cancer, hormone positive breast cancer, and HER2 negative breast cancer, or a combination of one or more of the foregoing cancers.

In one embodiment, examples of “cancer” when used herein in connection with the present invention include cancer selected from castration-sensitive prostate cancer and castration-resistant prostate cancer or a combination of one or more of the foregoing cancers.

In one embodiment of the invention, the cancer is a solid tumor.

In one embodiment of the invention, the cancer is a solid tumor which solid tumor is androgen-dependent.

In one embodiment of the invention, the cancer is a solid tumor which solid tumor expresses androgen receptors.

In one embodiment, the cancer is prostate cancer.

In one embodiment, the cancer is high-risk prostate cancer.

In one embodiment, the cancer is locally advanced prostate cancer.

In one embodiment, the cancer is high-risk locally advanced prostate cancer.

In one embodiment, the cancer is castration-sensitive prostate cancer.

In one embodiment, the cancer is metastatic castration-sensitive prostate cancer.

In one embodiment, the cancer is castration-sensitive prostate cancer or metastatic castration-sensitive prostate cancer with DNA damage repair mutations (DDR mutations). The DDR mutations include ATM, ATR, BRCA1, BRCA2, CHEK2, FANCA, MLH1, MRE11A, NBN, PALB2, and RAD51C.

In one embodiment, the cancer is hormone sensitive prostate cancer, also known as castration sensitive prostate cancer. Hormone sensitive prostate cancer is usually characterised by histologically or cytologically confirmed adenocarcinoma of the prostate which is still responsive to androgen deprivation therapy.

In one embodiment, the cancer is non-metastatic hormone sensitive prostate cancer.

In one embodiment, the cancer is high risk, non-metastatic hormone sensitive prostate cancer.

In one embodiment, the cancer is metastatic hormone sensitive prostate cancer.

In one embodiment, the cancer is castration-resistant prostate cancer, also known as hormone-refractory prostate cancer or androgen-independent prostate cancer. Castration resistant prostate cancer is usually characterised by histologically or cytologically confirmed adenocarcinoma of the prostate which is castration resistant (for example defined as 2 or more consecutive rises of PSA, week between each assessment, optionally resulting in 2 or more 50% or greater increases over the nadir, with PSA level ≥2 ng/mL), in a setting of castrate levels of testosterone (for example 1.7 nmol/L level of testosterone or ≤50 ng/dL level of testosterone), which castrate levels of testosterone are achieved by androgen deprivation therapy and/or post orchiectomy.

In one embodiment, the cancer is non-metastatic castration-resistant prostate cancer.

In one embodiment, the cancer is non-metastatic castration-resistant prostate cancer.

In one embodiment, the cancer is metastatic castration-resistant prostate cancer.

In one embodiment, the cancer is metastatic castration-resistant prostate cancer with DNA repair deficiencies.

In one embodiment, the cancer is breast cancer.

In one embodiment, the cancer is locally advanced or metastatic breast cancer.

In one embodiment, the cancer is triple negative breast cancer.

In one embodiment, the cancer is hormone positive breast cancer, including estrogen positive and/or progesterone positive breast cancer.

In one embodiment, the cancer is HER2 negative breast cancer.

In one embodiment, the cancer is germline BRCA-mutated HER2-negative breast cancer.

In one embodiment, the cancer is HER2 positive breast cancer.

In one embodiment, the cancer is triple positive breast cancer.

In one embodiment, the cancer is ovarian cancer.

In one embodiment, the cancer is small cell lung cancer.

In one embodiment, the cancer is Ewing's sarcoma.

In one embodiment, the cancer is hepatocellular carcinoma.

In one embodiment, the cancer is salivary gland tumor.

In one embodiment, the cancer is locally advanced.

In one embodiment, the cancer is non-metastatic.

In one embodiment, the cancer is metastatic.

In one embodiment, the cancer is refractory.

In one embodiment, the cancer is relapsed.

In one embodiment, the cancer is intolerable of standard treatment.

In one embodiment, the cancer has a CDK12 mutation.

In one embodiment of the present invention, the method is administered to a subject diagnosed with cancer, which cancer has developed resistance to treatment.

In a further aspect, the methods of the present invention may additionally comprise administering further anti-cancer agents, such as anti-tumor agents, anti-angiogenesis agents, signal transduction inhibitors and antiproliferative agents, which amounts are together effective in treating said cancer. In some such embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, androgen deprivation therapy and anti-androgens. In an embodiment of the present invention, the further anti-cancer agent is an anti-androgen. In an embodiment of the present invention, the anti-androgen is enzalutamide or apalutamide.

### Example 1: Genomic Analysis from a Phase II Trial of Talazoparib in BRCA1/2 Wild-Type HER2-Negative Breast Cancer and Other Solid Tumors: Homologous Recombination (HR) Deficiency Scores, Loss-of-Heterozygosity and Mutations in Non-BRCA1/2 Mutant Tumors with Other HR Mutations

### Methods

### Clinical Trial Design:

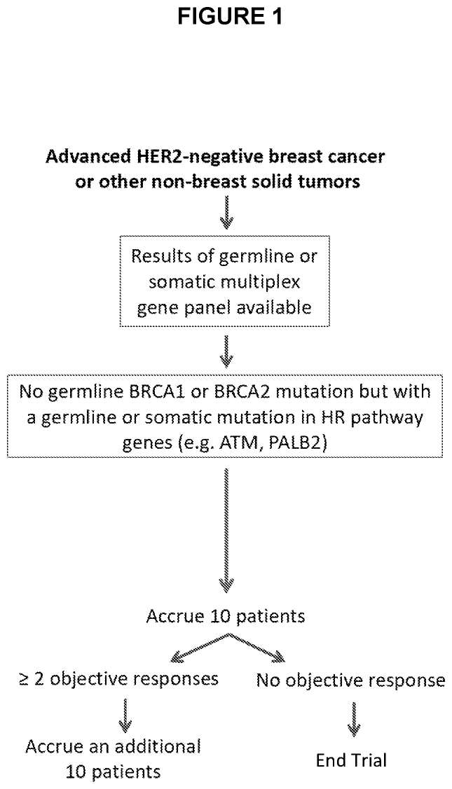

A phase II clinical trial of the PARP inhibitor talazoparib in BRCA1 and BRCA2 wild-type patients with HER2-negative advanced breast cancer or other solid tumors with a mutation in homologous recombination was conducted as shown in FIG. 1.

### Eligibility Criteria:

Eligible patients were adults (≥18 years old) with HER2-negative advanced or metastatic breast cancer that had progressed on at least 1 prior line of therapy for metastatic disease. Eligible patients also included adults (≥18 years old) with advanced or metastatic solid tumors beyond breast cancer that had progressed on at least one prior line of therapy. Patients were required to have no pathogenic mutations in either BRCA1 or BRCA2 genes on germline or somatic testing. They were required to have a pathogenic or likely pathogenic mutation in a HR-pathway associated gene detected on multiplex germline or somatic testing. These genes include: PALB2, CHEK2, ATM, NBN, BARD1, BRIP1, RAD50, RAD51C, RAD51D, MRE11, ATR, PTEN, Fanconi anemia complementation group of genes (FANCA, FANCC, FANCD2, FANCE, FANCF, FANCG, FANCL), plus other HR-related genes at the discretion of the primary investigators. Measurable disease by RECIST version 1.1 was required. There was no upper limit on the number of prior systemic therapies allowed prior to study entry. Patients were not allowed to have progressed during therapy with a platinum agent or within 8 weeks of discontinuing a platinum agent. Subjects were required to have an Eastern Cooperative Oncology Group (ECOG) performance status (PS) of 0-2 and adequate organ function based on screening laboratory values of liver, renal and hematologic parameters. The ability to take oral medications was required. Sexually active patients of childbearing potential were mandated to use contraception and females of childbearing age were tested for pregnancy at screening; females were not permitted to participate if pregnant or nursing and were tested for pregnancy at screening.

Patients were excluded if they previously took any PARP inhibitor, had taken any anti-cancer therapy within 21-days of study entry, received radiation therapy within 14 days of study entry, had active brain metastasis requiring treatment or leptomeningeal disease. Patients with human immunodeficiency virus infection or active hepatitis C or hepatitis B viral infections were excluded. Also, patients requiring prolonged hospitalization, major surgery or receiving other investigational agent within 21 days prior to study entry were excluded. Additional key exclusion criteria were: other medical co-morbidity likely to interfere with study participation eg. infection grade ≥3 CTCAE version 4, or requiring parenteral antibiotics within 7 days, or known hypersensitivity to talazoparib.

### Treatment:

Talazoparib was administered at 1 mg orally daily continuously, taken whole, at approximately the same time each day. Treatment cycles lasted 28 days. Patients were evaluated on day 1 of each treatment cycle by physician assessment including medical history, physical examination, laboratory values (complete blood count (CBC) and comprehensive metabolic panel), vital signs, ECOG PS assessment and urine pregnancy test (for women of childbearing age only). Day 14 physician assessment was required for the first cycle and CBCs were required weekly for the first cycle, then prior to start of each subsequent cycle. Treatment continued until disease progressed or unacceptable toxicity. Safety was assessed at each physician visit and monitored continuously by laboratory values, patient reporting and patient diary. Adverse events (AEs) and severe AEs (SAEs) were graded according to CTCAE version 4.0. SAEs grade ≥3 were reported to the Data Safety Monitoring Committee.

### Evaluation of Tumor Responses:

Tumor evaluation was performed at baseline and after every two cycles and responses were assessed according to RECIST version 1.1. After six cycles, tumor evaluations were allowed every 3 cycles per physician discretion. CT scan was required at baseline and patients with known or suspected bone disease were required to have bone imaging (eg. bone scan or PET scan) at baseline and subsequent tumor evaluations.

### Tumor Genomics:

HRD scores were assessed for Formalin-Fixed Paraffin-Embedded (FFPE) tumor tissues by MyChoice® CDx HRD assay (Myriad). Next generation sequencing of FFPE tumor tissue was performed using a 108 gene panel assay (Myriad).

### Statistical Analysis:

All 20 patients enrolled were included in the analysis. Tumor responses were categorized as complete response (CR), partial response (PR), stable disease (SD), or progressive disease (PD) by RECIST version 1.1. The primary objective was the objective response rate (ORR) and secondary objectives included: clinical benefit rate (CBR: CR+PR+SD), PFS, and safety. The statistical plan was designed with a null hypothesis of an ORR≤5%, and was powered to 80% to detect an ORR≥30% with an alpha of 0.05. Based on statistical constraints, if at least 3 patients out of 20 respond, statistical significance will be declared.

### Results

### Patient Characteristics:

Twenty patients were enrolled in this two-stage study based on identification of a non-BRCA1 or BRCA2 HR-associated mutation in a next-generation sequencing assay of either germline or tumor tissue. Based on two partial responses observed in the first stage of 10 patients, an additional 10 patients were enrolled according to the study design (FIG. 1). Of the twenty patients enrolled, thirteen patients had HER2-negative breast cancer (n=11 hormone receptor positive; n=2 triple negative breast cancer (TNBC)) and 7 patients had other tumor types (n=3 pancreas, n=1 each of mixed Mullerian uterine, testicular, parotid acinic cell carcinoma). Seventy-five percent of patients were female with a median age of 53.9 years. Patients had received a median of 2 prior lines of therapy for advanced disease (range 1-8). Prior lines of therapy included chemotherapies, hormonal therapies and targeted agents. Platinum-based therapies had been previously administered to 35% of patients, but patients with disease progression within 8 weeks of last platinum dose were excluded from this study.

Enrolled patients had germline mutations in ATM (n=3), BRIP1 (n=2), CHEK2 (n=3), FANCA (n=1), PALB2 (n=6) or somatic mutations in ATM (n=2), ATR (n=1), PTEN (n=5), RAD50 (n=1) as detected by any CLIA-approved next-generation sequencing assay performed on either germline tissue or tumor tissue (Table 1). Mutations were required to have a clinical annotation of pathogenic or likely-pathogenic. Two patients had multiple qualifying mutations at the time of study enrollment (pancreas cancer with gPALB2/gBRIP1; breast cancer with gCHEK2/gFANCA/sPTEN).

TABLE 1
Germline (n = 15) or Somatic (n = 9) Mutations Identified
by Next-Generation Sequencing used for Enrollment

| Mutation | Germline (n = 15) | Somatic (n = 9) |
|---|---|---|
| ATM | 3 | 2 |
| ATR | 0 | 1 |
| BRIP1 | 2 | 0 |
| CHEK2 | 3 | 0 |
| FANCA | 1 | 0 |
| PALB2 | 6 | 0 |
| PTEN | 0 | 5 |
| RAD50 | 0 | 1 |

### Talazoparib Efficacy:

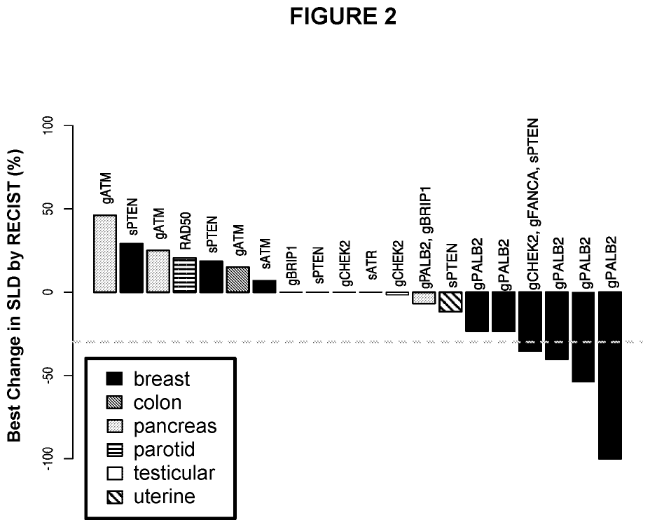

All enrolled patients were treated with talazoparib monotherapy at 1 mg orally daily. Nineteen patients discontinued therapy due to disease progression; one patient withdrew from therapy with RECIST stable disease due to concern of non-target disease enlargement. Response rates as documented by RECIST version 1.1 were stratified by breast cancer and non-breast cancer groups (Table 2). Best treatment responses were summarized by a waterfall plot for all twenty patients treated in the study (FIG. 2).

TABLE 2
Best Response as Documented by RECIST v.1.1.

| | Response Rate, n (%) | | |
|---|---|---|---|
| | Breast Cancer | Non-Breast | Combined |
| Best Response | (N = 13) | Cancer (N = 7) | (N = 20) |
| Complete Response (CR) | 0 (0%) | 0 (0%) | 0 (0%) |
| Partial Response (PR) | 4 (31%) | 0 (0%) | 4 (20%) |
| Stable Disease (SD) | 6 (46%) | 4 (57%) | 10 (50%) |
| Progressive Disease (PD) | 3 (23%) | 3 (43%) | 6 (30%) |
| ORR (CR + PR) | 4 (31%); | 0 (0%); | 4 (20%); |
| | 95% | 95% | 95% |
| | CI: 9-61% | CI: 0-41% | CI: 6-44% |
| CBR (CR + PR + SD ≥ | 7 (54%); | 2 (29%); | 9 (45%); |
| 6 mos) | 95% | 95% | 95% |
| | CI: 21-81% | CI: 37-71% | CI: 23-68% |

n = number of patients with response; N = number of patients per cohort.

### Evaluation of Tumor HRD Score as a Biomarker for Talazoparib Response:

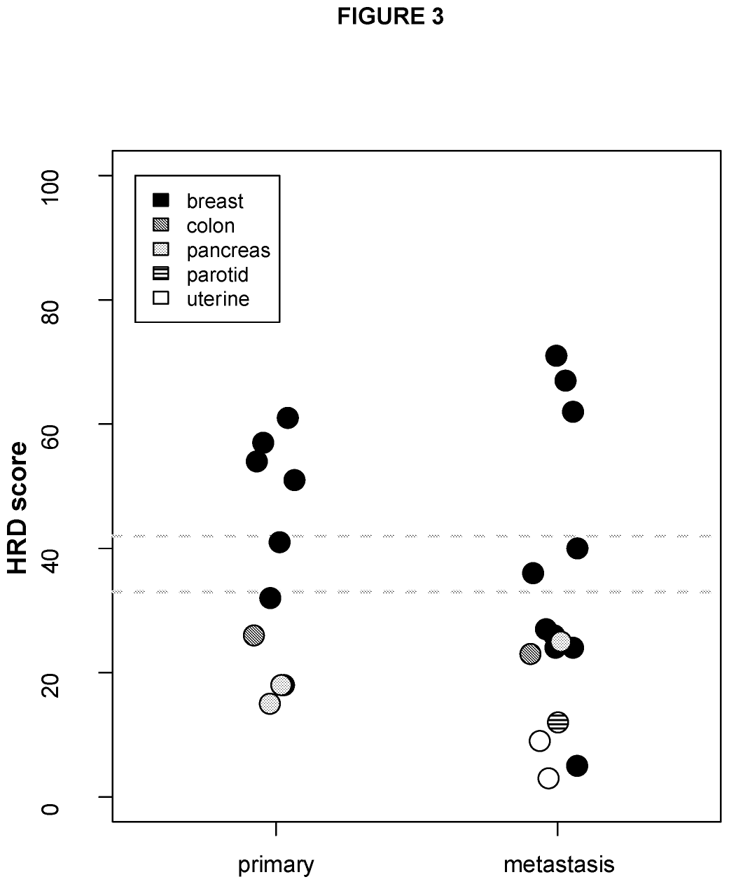

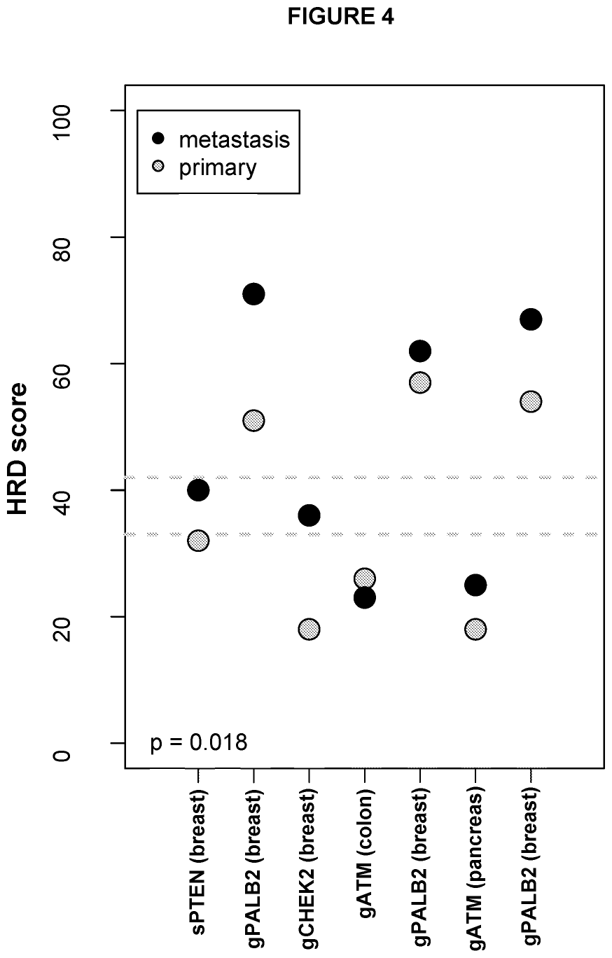

To determine whether the tumors from patients enrolled on this study had high levels of genomic instability, the Myriad MyChoice HRD assay (FIG. 3) was conducted on primary (n=12) or metastatic (n=17) FFPE tumor tissue of 18 of the 20 patients treated on this trial (2 excluded for insufficient sample). Of the 18 assays performed, 2 failed and were thus excluded from analysis. Seven patients had HRD analysis performed on both primary and metastatic tumor specimens (FIG. 4). For these patients the HRD score was significantly higher in the metastasis versus the biopsy (means 46.2 versus 36.5, p=0.018 by paired t-test). Thus, HRD scores were readily obtainable from archival FFPE specimens and the metastatic biopsies yielded higher HRD scores compared to the primary tumor.

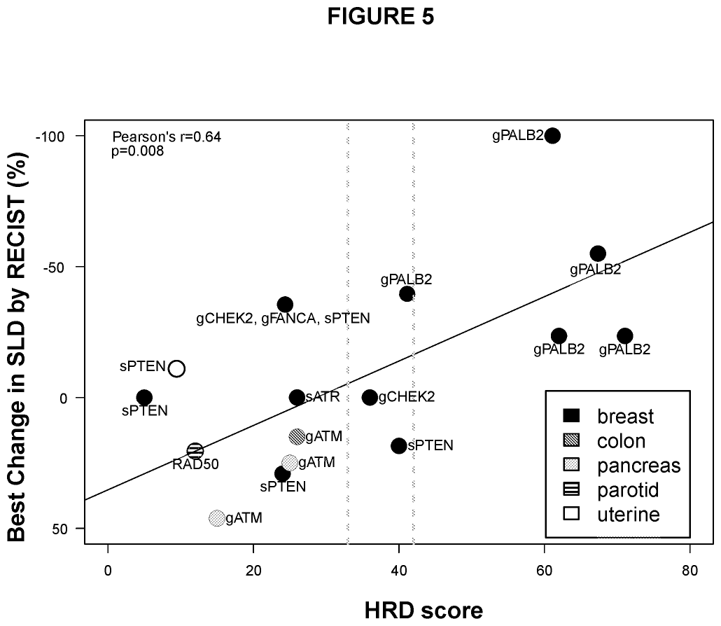

Next, to determine whether HRD scores could serve as a biomarker for response to talazoparib therapy, the best overall treatment response by change in the sum of the longest diameter of the target lesions (SLD) was plotted as a function of the tumor HRD score (FIG. 5). In cases where more than one HRD score was available per patient the higher score was used (for example, if both primary and metastatic scores were obtained). A positive correlation between treatment response and HRD score with higher HRD scores associated with better response to therapy (Pearson's r=0.64, p=0.008) was demonstrated. In particular, all 5 assayed tumors derived from patients with gPALB2 (1 gPALB2 tumor HRD score failed) passed the HRD cutoff of 33 and 4 out of 5 passed the HRD cutoff of 42. Thus, HRD score may be a useful biomarker for response to talazoparib monotherapy. Further, tumors with gPALB2 mutations were associated with a high degree of genomic instability that mirrored gBRCA1/2 mutated tumors.

As increased genomic instability was positively correlated with treatment response to talazoparib, further interrogation of genomic mutations in these tumors was performed. Primary and metastatic samples were sequenced with a hybridization-capture panel of 108 genes associated with HR-deficiency in human cancers. Genomic mutations in primary and metastatic lesions were binned. The most common alterations detected included mutations in PIK3CA (n=8), PALB2 (n=6), ATM (n=5), KRAS (n=4), PTEN (n=5) and TP53 (n=4). In all cases except one, the HR-associated mutation detected by CLIA-approved NGS used as entry criteria were detected (sRAD50 in the parotid tumor was not detected). This included all gPALB2, gCHEK2, and gATM mutations used as entry criteria. In addition, all sPTEN mutations used as entry criteria were also detected. The findings indicate that these alterations were likely to be present in a high allelic fraction of the sampled tumors and therefore likely contributed to either disease onset or malignant progression.

Finally, the NGS panel assay was utilized to detect LOH at the assayed genes (Table 3).

TABLE 3
Loss of Heterozygosity (LOH) Analysis from Tumor Sequencing.

| | | LOH | Secondary | |
|---|---|---|---|---|
| Mutation | n | detected | mutations | Other |
| gATM | 3 | 1 | 2 | |
| gBRIP1 | 2 | | | i, u |
| gCHEK2 | 3 | 3 | | |
| gFANCA | 1 | 1 | | |
| gPALB2 | 6 | 3 | 2 | i |
| sATM | 2 | 1 | 1 | |
| sATR | 1 | 0 | | |
| sPTEN | 5 | 1 | 2 | u, f |
| sRAD50 | 1 | | | nd |
| sTP53 | 4 | 4 | | |
| sRB1 | 3 | 3 | | |

n = number of mutations detected. Secondary mutations included either deleterious SNV, frameshift mutation or large-scale rearrangement. i = insufficient sample, u = uncertain, f = failed, nd = not detected on follow-up tumor sequencing.

For the tumors with gPALB2 mutations, 3 of the 6 had LOH for PALB2, and an additional two tumors had 2 independent PALB2 mutations suggesting bi-allelic inactivation. The one remaining tumor had an uncertain LOH result in the setting of the failed HRD assay for that specimen. Thus, it is likely that most, if not all, of the tumors in the cohort with gPALB2 mutations had complete inactivation of PALB2 gene function. Other detected mutations that were associated with LOH included all s TP53 mutations (n=4), all gCHEK2 mutations (n=3), gFANCA (n=1), all sRB1 mutations (n=3) and NF1 (n=1). Of the three gATM mutations one had LOH, while the others (n=2) had two independent mutations, suggestive of bi-allelic inactivation. sATM mutations were associated with LOH in a breast cancer and with 2 independent (possibly bi-allelic) mutations in testicular cancer. Thus, multiple genes associated with HR-deficiency were likely associated with LOH and/or bi-allelic inactivation in tumors, especially gPALB2, gCHEK2, and gATM/sATM.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1. A method of identifying a metastatic tumor determined to have a mutation in homologous recombination pathway genes, that is sensitive to treatment with talazoparib, or a pharmaceutically acceptable salt thereof, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

2. The method of claim 1, wherein the mutation is a somatic or a germline mutation.

3. The method of claim 1, wherein the mutation is PALB2, CHEK2, ATM, BR1P1, RAD50, ATR, PTEN, or FANCA.

4. The method of claim 1, wherein the mutation is gCHEK2, gPALB2, or sPTEN.

5. The method of claim 1, wherein the mutation is gPALB2.

6. The method of claim 1, wherein the metastatic tumor is a breast tumor.

7. The method of claim 6, wherein the breast tumor is a HER2-negative breast tumor.

8. The method of claim 1, wherein the metastatic tumor is determined to have a mutation in homologous recombination pathway genes by next generation sequencing.

9. The method of claim 1, wherein the step of determining a homologous recombination deficiency score from a biopsy of the metastatic tumor is performed by next generation sequencing.

10. The method of claim 1, wherein the homologous recombination deficiency score is at least 42%.

11. A method of treating metastatic cancer comprising administering talazoparib, or a pharmaceutically acceptable salt thereof, according to claim 1.

12. A method of treating metastatic cancer in a subject determined to have a mutation in homologous recombination pathway genes, comprising a) determining a homologous recombination deficiency score from a biopsy of the metastatic tumor; and b) administering talazoparib, or a pharmaceutically acceptable salt thereof, if the homologous recombination deficiency score is at least 33%, wherein the mutation is not germline BRCA1 or germline BRCA2.

13. The method of claim 12, wherein the mutation is a somatic or a germline mutation.

14. The method of claim 12, wherein the mutation is PALB2, CHEK2, ATM, BRIP1, RAD50, ATR, PTEN, or FANCA.

15. The method of claim 12, wherein the mutation is gCHEK2, gPALB2, or sPTEN.

16. The method of claim 12, wherein the mutation is gPALB2.

17. The method of claim 12, wherein the metastatic tumor is a breast tumor.

18. The method of claim 17, wherein the breast tumor is a HER2-negative breast tumor.

19. The method of claim 12, wherein the metastatic tumor is determined to have a mutation in homologous recombination pathway genes by next generation sequencing.

20. The method of claim 12, wherein the step of determining a homologous recombination deficiency score from a biopsy of the metastatic tumor is performed by next generation sequencing.

21. The method of claim 12, wherein the homologous recombination deficiency score is at least 42%.
